(19) United States
(12) Patent Application Publication
(10) Pub. No.: US 2019/0195853 A1
(43) Pub. Date: Jun. 27, 2019

(54) METHOD AND SYSTEM FOR YARN QUALITY MONITORING

(71) Applicants: Petr PERNER, Cesky Dub (CZ); Ondrej Zelinka, Cesky Dub (CZ); Josef Suska, Simonovice (CZ)

(72) Inventors: Petr PERNER, Cesky Dub (CZ); Ondrej Zelinka, Cesky Dub (CZ); Josef Suska, Simonovice (CZ)

(21) Appl. No.: 15/854,014

(22) Filed: Dec. 26, 2017

(51) Int. Cl. G01N 33/36; G06T 7/00; G06T 7/90; G01N 21/952; G01N 21/88; G02B 27/42

(52) U.S. Cl. CPC G01N 33/365; G06T 7/0004; G06T 7/90; G01N 21/952; G01N 21/8806; G01N 21/8851; G01N 2021/8835; G06T 2207/10024; G06T 2207/10048; G06T 2207/30108; G01N 2201/0634; G01N 2021/8887; G01N 2021/8845; G02B 27/4205

(57) ABSTRACT

There is disclosed a method and an apparatus for monitoring textile yarn quality. Textile yarn is checked for quality to meet the required criteria such as diameter evenness and unwanted foreign fiber presence by utilizing an artificial diffuse light illuminating image sensor, with the yarn placed as an obstacle, into the pathway of the light, the contours of the light being focused for sharp image capture. The yarn diameter is determined by processing of focused yarn image.

### RELATED APPLICATION

This application is related to commonly owned U.S. patent application Ser. No. ______, entitled: METHOD AND DEVICE FOR OPTICAL YARN QUALITY MONITORING, Attorney Docket No. 6784/2, filed on even date herewith.

### FIELD OF THE INVENTION

The invention relates to a method and device for diameter measuring and color inspection of a linear textile formation such as yarn, thread, textile fibers and slivers.

### BACKGROUND OF THE INVENTION

Yarn is a textile formation made by spinning of a relatively short fibrous material into a long continuous unit. Raw cotton is used as one of the many input materials for yarn production. Machine yarns are wound onto coils for easy storage and further processing.

Yarn evenness and foreign fibers are important quality parameters in yarn. Yarn evenness refers to the variation in diameter along its length, as yarn has small changes in diameter along its length. There are numerous locations of thick and thin portions along the yarn length. This affects evenness of the yarn.

Foreign fiber is the presence of foreign particles in the textile yarn. Textile yarn is generally white in color. Foreign fibers interfere with color homogeneity of the yarn. Yarn color analysis determines the presence of foreign fibers. Yarn evenness and foreign fibers are affected by quality of the materials used. Yarn evenness is also affected during manufacturing by spinning machines. As yarn is produced rapidly and in high volumes, manual inspection by human inspectors is not feasible, but rather, is impractical, and not possible.

### SUMMARY OF THE INVENTION

The present invention provides methods and apparatus, e.g., systems and devices, for monitoring yarn quality for measuring yarn diameter and detecting foreign fibers. The present invention is particularly suitable for mass machine production of yarn, which has increased resistance against accumulated dust particles and which is easily realizable.

Embodiments of the invention provide methods for yarn quality monitoring, which are carried out by a primary artificial light source directly illuminating an image sensor through a focusing element. Longitudinally moving yarn is placed as an obstacle between the light source and the image sensor. Images of yarn contours are focused on the image sensor. The longitudinally moving yarn is guided by guiding elements, and is split into small consecutively measured yarn segments whose longitudinal dimension is defined by a longitudinal dimension of a photosensitive area of the image sensor, the speed of yarn movement, and the image sensor sampling rate. Image sensor data for each measured segment is evaluated and the diameter of the yarn is calculated.

Embodiments of the invention provide a setup of the yarn, to create an obstacle in the diffused light beam, such that the focusing element creates images with sharp yarn contours suitable for accurate determinations of yarn diameter. By using the focusing element, the effects of dust particles accumulated on both optical surfaces (emitter and receiver) placed out of focus are eliminated. Additionally, unwanted ambient light rays coming from the sides are directed outward from the image sensor by the focusing element.

In embodiments of the invention, two types of light sources are used: a primary light source, which directly illuminates an image sensor, and, a secondary light source, whose rays are reflected from the yarn before they reach the image sensor.

In other embodiments of the invention, a diffraction element is used for diffracting at least portion of light rays. Diffracted light rays create an interference pattern on the image sensor. Undiffracted light rays, originating from the primary light source create a yarn contour image on the image sensor (the “yarn contour image” or “contour image” is created from yarn shadows and nonblocked primary light source rays). Undifracted light rays originating from the secondary light source (reflected from the yarn), create a yarn image on the image sensor. Image sensor captured data is created as any combination of yarn contour image, yarn image and interference patterns.

Embodiments of the present invention eliminate problems of the conventional art, where dust particles and fibers on optical surfaces create sharp shadows on the image sensor. These shadows make accurate yarn diameter evaluation impossible. Embodiments of the present invention are such that particles accumulated on the optical surfaces do not create shadows on the image sensor, even if its size is comparable with the measured yarn itself. Only light intensity on the image sensor will be lower. However, this effect is compensated for by increasing power of the primary artificial light source. Another advantage of using direct image sensor illumination is that significantly lower power of the primary artificial light source is needed to excite the image sensor pixels, than that for configurations where reflected light is captured for yarn diameter processing.

In another embodiment of the present invention, measured parts of yarn are illuminated by a secondary artificial light simultaneously with the illumination from the primary artificial light source. The secondary artificial light source is placed on same side of yarn as the image sensor. Light rays from the secondary light source are reflected from the moving yarn and are focused on the image sensor by a focusing element. Secondary artificial light rays reflected by the yarn are diffracted by a diffraction element (placed in front of focusing element or in front of the image sensor), and an interference pattern is created. The focusing element significantly improves image quality. Images of moving yarn contours and its respective interference pattern are simultaneously captured by the image sensor for further diameter measurements and color analysis.

Embodiments of the invention are such that when the diffraction element is employed, the color image sensor is not necessary, and therefore, not used, such that only the monochromatic image sensor is used. Measured yarn segment color information is obtained by the processing of light intensity at corresponding pixel positions on the image sensor. Corresponding pixel position depends on color wavelength, yarn diameter, and yarn position. The monochromatic image sensor has high sensitivity, high resolution, while being inexpensive to use.

Embodiments of the invention are such that when using a diffraction element with a zero order mode feature, part of the light rays pass undiffracted through the diffraction element, and are focused on the image sensor as a yarn image or yarn contour image. The interference pattern created by the diffracted rays is obtained by being captured simultaneously with the yarn image and/or the yarn contour image by the image sensor. Color analysis is obtained by processing of captured interference pattern and yarn diameter is obtained by processing of captured yarn contour image and/or yarn image.

For yarn quality monitoring, color analysis and yarn diameter are obtained from the same yarn segment. This is in contrast to the contemporary art, where yarn segment data for color analysis and yarn segment data for yarn diameter calculations are captured by separate independent light detectors, which require data synchronization, depending on the speed at which the yarn is moving.

Embodiments of the invention are such that monochromatic light, RGB (Red-Green-Blue) light, white light, UV (Ultra-Violet) light, and IR (Infra-Red) light is used as the secondary artificial light source. This secondary artificial light source illuminates the yarn, and the image sensor captures light rays reflected from the yarn. Specific light wavelengths are suitable for foreign fiber detection and monitoring of various yarn materials, e.g. cotton, wool, or synthetic materials such as polypropylene.

Embodiments of the invention are such that the primary artificial light uses wavelengths different from secondary artificial light. Such wavelength selection in combination with optical filter, which partially covers the image sensor, allows the use of primary artificial light rays only to measure the diameter of the yarn.

In other embodiments of the invention, parameters of primary artificial light and/or the secondary artificial light source are controlled via feedback. The feedback is based on the quality of previously captured images. For example, light intensity is increased when images with low light intensity were previously captured.

Embodiments of the invention are directed to an apparatus in the form of a device for yarn quality monitoring. The device includes at least one primary artificial light source illuminating the image sensor, at least one focusing element, at least one image sensor, and a control unit for processing image sensor data.

In another embodiment of the invention, the device includes a primary artificial light source, which emits diffusive light, or an additional light diffuser is used. The device also includes at least one optical element in an optical path of primary artificial light for focusing of a yarn contour image on an image sensor.

In other embodiments of the invention, in the device, a secondary artificial light source for illuminating the yarn and diffraction element is added.

In other embodiments, the diffraction element is a diffraction grating. The advantage of the diffraction grating is simplicity and economics.

Other embodiments of the device employ monochromatic light, RGB light, white light, UV light, IR light, as a secondary artificial light source. Secondary artificial light source illuminates the yarn and the image sensor captures light reflected from the yarn. Specific light wavelengths or its combination, are suitable for detection and monitoring of various yarn materials, e.g. cotton, wool, or synthetic materials, such as polypropylene.

Other embodiments of the invention utilize a primary artificial light source with wavelengths different from the secondary artificial light source. Such wavelength selection in combination with an optical filter, covering partially the image sensor, allows for the use of the primary artificial light source only as a single source for measuring the diameter of the yarn.

In another embodiment, the device includes sources of primary and/or secondary artificial light, which are controlled via feedback, based on the quality of previously captured images. For example, light intensity is increased when images with low light intensity were previously captured.

In other embodiments of the device according to the invention, the focusing element is formed by at least one lens or a combination of lenses, from the group of aspherical lens, mirror lens, convex lens, and concave lens. Lenses or combinations of lenses can be mounted in M12×0.5 package. The size and pitch of M12×0.5 package allows for the adjustment of a focus distance, and the M12×0.5 package is available at low costs.

In other embodiments of the device, the image sensor is formed by at least one line or array of light-sensitive pixels. Individual pixels or regions of pixels can be covered with an optical filter layer for specific ranges of light wavelengths. This simplifies further processing of the captured images for diameter measurements and yarn color analysis.

In other embodiments of the device according to the invention, the image sensor pixels can include micro lenses for increased sensitivity and limiting of wide angle light rays, such that the image is made sharper.

In another embodiment of the device according to the invention, the image sensor is a charge coupled device (CCD) type. CCD image sensors are used, as they have high sensitivity and are low cost.

In another embodiment of the device according to the invention, the image sensor is a complementary metal oxide semiconductor (CMOS) type. CMOS image sensors have high speed and low power consumption.

In another embodiment of the device according to the invention, the control unit is provided with a field-programmable gate array (FPGA) or programmable logic device (PLD) programmable logic array (PLA), or application specific integrated circuit (ASIC) integrated circuit, for fast parallel image data processing, microcontroller for yarn quality monitoring, light source control circuits and power supply.

In another embodiment of the device according to the invention, an ASIC can be used to integrate image sensor with data processing on one chip.

The present invention is advancement over the contemporary art, as it allows for accurate yarn measurements and color analysis when significant amounts of dust particles are present. Additionally, the invention provides methods and devices able to measure yarn diameters and detect foreign fibers from the same image, as captured by an image sensor.

Embodiments of the invention are directed to a yarn quality monitoring device or system. The device comprises: a primary light source (e.g., a primary artificial light source) for emitting light, the light propagation (e.g., travel) defining an optical path; a diffuser proximate to the first light source for diffusing the emitted light; an image sensor illuminated by the diffused light emitted from the primary light source, for capturing a yarn contour image from illumination of a moving yarn in the optical path; a focusing element in the optical path between the first light source and the image sensor, for focusing the yarn contour image on the image sensor; and, a controller in communication with the image sensor for processing the captured yarn contour image, to determine yarn diameter

Optionally, the device additionally comprises: a secondary light source (e.g., a secondary artificial light source) for illuminating the yarn, the reflected light propagation defining an optical path; a diffraction element in the optical path, between the yarn and the image sensor, the diffraction of secondary light source light reflected from the yarn for interference pattern creation on the image sensor; and, the controller is also configured for processing the interference pattern to obtain yarn color analysis.

Optionally, the diffraction element is a diffraction grating.

Optionally, the primary light source and the secondary light source emit light at different wavelengths, and the device further includes an optical filter at least partially covering the image sensor for blocking light reflected from the yarn illuminated by secondary light source.

Optionally, the secondary light source emits one or more light types including monochromatic light, RGB (Red-Green-Blue) light, white light, UV (ultraviolet light) or IR (infra-red) light.

Optionally, the focusing element includes one or more lenses such as aspherical lenses, mirror lenses, convex lenses, concave lenses and combinations thereof.

Optionally, the focusing element includes one or more lenses in an M12×0.5 package.

Optionally, the image sensor comprises: one or more lines of photosensitive pixels; and, optionally, the image sensor is type of a CMOS (complementary metal oxide semiconductor) sensor or a CCD (charge coupled device).

Optionally, the image sensor includes microlenses configured to limit wide angle light rays.

Optionally, the controller comprises: a programmable logic array (PLA) for image data processing, to obtain yarn diameter and/or yarn color analysis; a light control circuit in communication with the PLA, which is controlled by the PLA; and, a microcontroller in communication with the PLA, the microcontroller programmed to evaluate the yarn eveness, and/or the yarn foreign fiber content, from the processed image data.

Embodiments of the invention are directed to a method of yarn quality monitoring. The method comprises: illuminating an image sensor with light from a primary light source configured for creating a contour image of a yarn on the image sensor, the primary light source diffused by a diffuser and focused by a focusing element, on an image sensor, the primary light source, diffuser and focusing element aligned to define an optical path; obtaining a contour image of the yarn by the image sensor, the moving yarn positioned in the optical path between the primary light source and the image sensor; and, processing the yarn contour image to obtain a yarn diameter.

Optionally, the method further comprises: illuminating the moving yarn by a secondary light source, such that the light from the secondary light source is reflected from the moving yarn; focusing the reflected light on the image sensor; diffracting the reflected light from the moving yarn to cause the creation of an interference pattern on the image sensor; obtaining the interference pattern from the image sensor; and, processing of the interference pattern for obtaining a yarn color analysis.

Optionally, the method is such that the secondary light source emits at least one type of light including: monochromatic light, RGB light, white light, UV light, IR light, and combinations thereof.

Optionally, the method is such that the illuminating by the primary light source is at a wavelength different from a wavelength of light emitted from the secondary light source, and optionally, the method further comprises: covering a portion of the image sensor with an optical filter; passing the emitted light from the primary light source through the optical filter to a portion of the image sensor; and, blocking light reflected from the yarn, to cause illumination from only the primary light source for measuring a diameter of the moving yarn.

Optionally, the method is such that the capturing of the contour image of the moving yarn and the capturing of the interference pattern is performed simultaneously by the image sensor.

Optionally, the method is such that it additionally comprises: controlling the primary light source to emit light at an intensity based on processing results from a previously captured image from the image sensor in a feedback loop.

Optionally, the method is such that it additionally comprises: controlling the secondary light source to emit light at an intensity based on processing results from previously captured image from the image sensor in a feedback loop.

Unless otherwise defined herein, all technical and/or scientific terms used herein have the same meaning as commonly understood by one of ordinary skill in the art to which the invention pertains. Although methods and materials similar or equivalent to those described herein may be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Additional objects and features of the present invention will become apparent upon reading of the detailed description of the invention and the appended claims provided below, and upon reference to the drawings in which:

FIG. 1 is a perspective view of a system in accordance with an embodiment of the invention;

FIG. 2 is a perspective view of a system in accordance with an embodiment of the invention;

FIG. 3 is a block diagram of the controller of FIGS. 1 and 2; and,

FIGS. 4A and 4B are flow diagrams of processes performed in the controller of the systems of FIGS. 1 and 2, for yarn evenness monitoring and foreign fiber detection.

### DETAILED DESCRIPTION OF THE INVENTION

It is understood that the below described and depicted particular cases of embodiments of the invention are presented for illustration and not to limit the invention to such examples. Those skilled in the art will find or will be able to provide, based on routine experimenting, one or more equivalents of the embodiments of the invention disclosed herein. Such equivalents shall be included into the scope of the following claims.

Embodiments of the present invention are directed to methods and systems for yarn quality monitoring, for example, for yarns used in the textile industry.

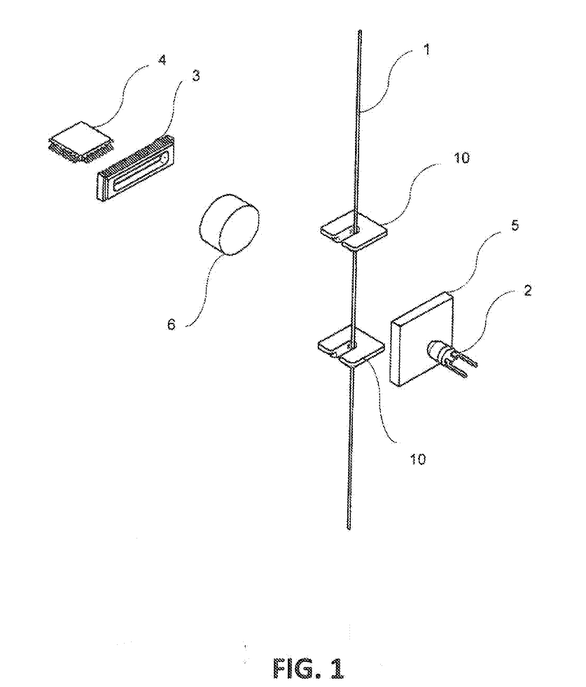

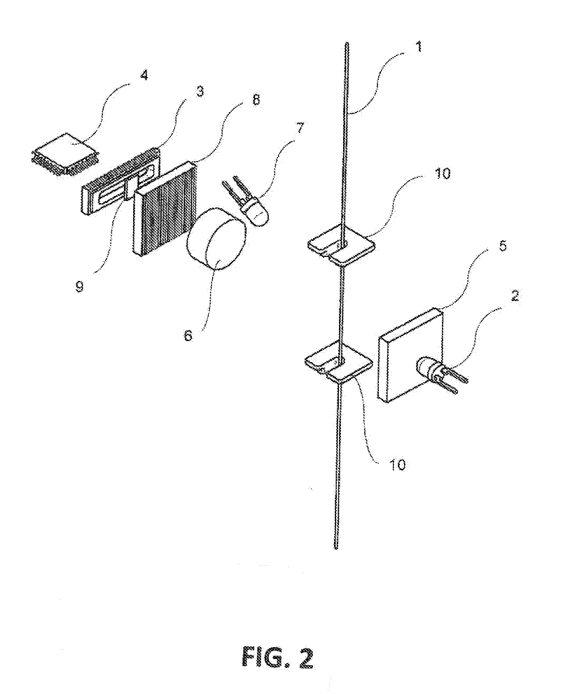

FIG. 1 shows a yarn quality monitoring system in an exemplary device, where a primary or first artificial light source 2 directly illuminates an image sensor 3, such that the light propagates, e.g., travels, along an optical path, through a focusing element 6 and diffuser 5. Guiding elements 10 stabilize longitudinally moving yarn 1 creating an obstacle for diffused light rays from the light source 2, which illuminate the image sensor 3. Turning also to FIG. 2, the focusing element 6 directs the non-blocked light rays through a diffraction element 8 making an image of yarn contours on the image sensor 3. A yarn contour image, also referred to herein as a contour image, for example, is an image created from a yarn, such as a moving yarn 1, by light from the primary light source 2 not blocked by the yarn, and the shadow(s) of the yarn. Yarn diameter is determined by processing of yarn contour image(s) on the image sensor 3, by the control unit or controller 4.

As shown in FIG. 2, a secondary or second artificial light source 7 is added to the system of FIG. 1. This secondary artificial light source 7 emits light at a wavelength different from primary artificial light source 2. This secondary artificial light source 7 is placed on the same side of the yarn 1 as the image sensor 3, and serves to illuminate the yarn 1. The secondary artificial light source 7 may be a plurality of the same or different artificial light sources 7. The emitted light reflected from the yarn 1 also propagates, e.g., travels, along the optical path to the image sensor 3. The secondary light source(s) 7 are used, for example, for detecting of various foreign fiber materials or to increase illumination intensity.

A diffraction element 8 serves to diffract at least a portion of secondary artificial light rays reflected from the yarn 1, which creates an interference pattern on the image sensor 3. For example, for white light, the diffraction element 8 creates rainbow spectra. The remaining portion of the reflected light rays passes through the diffraction element 8, without being diffracted, and creates a focused yarn image on the image sensor 3. This focused yarn image is, for example, used for diameter measurement of the yarn 1. For example, a diffraction grating can be used as standard and easily available diffraction element 8.

When the diffraction element 8 is employed, a color image sensor, as the image sensor 3 is not necessary, and therefore, not used, such that only a monochromatic image sensor, as the image sensor 3, is used. Measured yarn segment color information is obtained from a captured interference pattern by the processing of light intensity at corresponding pixel positions on the image sensor 3. A monochromatic image sensor, as the image sensor 3, allows for high sensitivity and high resolution.

When the diffraction element 8 has a zero order mode feature, a portion of the light rays pass undiffracted through the diffraction element 8, and are focused on the image sensor 3, as a yarn image and/or yarn contour image. The interference pattern created by the diffracted rays is obtained by being captured simultaneously with the obtaining by capturing the yarn image and/or yarn contour image by the image sensor 3, and is processed (in the controller 4) to obtain a color analysis, for determining foreign fibers in the yarn, e.g., foreign fiber content in the yarn, as well as a determination of yarn diameter. The yarn quality is monitored, such that color analysis and yarn diameter are obtained from the same yarn segment.

An optical filter 9, partially covers the image sensor 3, and allows for the passage of only primary artificial light source 2 rays, which are used for yarn 1 diameter measurement, while the optical filter blocks light rays from the secondary artificial light source 7. Interference pattern processing by the controller 4 is used to determine the presence of foreign fibers in the yarn 1. The yarn contour image and interference pattern are captured by the image sensor 3, contemporaneous in time, and for example, at the same time. For example, the interference pattern originates from the secondary light source 7, based on light rays reflected from the yarn, while the yarn contour image originates from the primary light source 2. Processing the light intensity of interference pattern at specific distances from the yarn 1 image on image sensor 3 can be used for color analysis of the yarn 1. For example, the detection of foreign fibers in the yarn 1 is achieved by a color image sensor, as the image sensor 3, but is not required, as a monochromatic image sensor need only be used as the image sensor 3.

The secondary light source 7 serves to illuminate the yarn contemporaneously, and, for example, simultaneously, with the illumination from the primary artificial light source 2. The secondary artificial light source 7 is placed on same side of yarn 1 as the image sensor 3. Light rays from the secondary light source 7 are reflected from the moving yarn 1 and are focused on the image sensor 3 by the focusing element 6. Secondary artificial light rays from the secondary light source 7 are reflected by the yarn 1 are diffracted by a diffraction element 8 (placed in front of focusing element 6 or in front of the image sensor 3). This results in an interference pattern created at the image sensor 3. The focusing element 6 improves image quality at the image sensor 3. Images of moving yarn contours and its respective interference pattern are simultaneously captured by the image sensor 3, for further diameter measurements and color analysis.

Should foreign fiber detection be desired, the secondary artificial light source 7 for illumination is necessary, to create an interference pattern, for example, through the diffraction element 8 (along with processes from the controller 4, as detailed below). The secondary artificial light source 7 is, for example, of specific wavelengths, such as those for monochromatic light, RGB light, white light, UV light, IR light or any combination thereof.

The focusing element 6 serves to eliminate the effects, such as distortion, caused by dust particles and other foreign and ambient particles, accumulated on optical surfaces (light sources 2, 7 and the focusing element 6). Additionally, the focusing element 6 directs unwanted lateral ambient light rays, directing these ambient light rays outward from the image sensor 3. The focusing element 6 is formed by a single lens or a set of lenses, the lenses including aspherical lenses, mirror lenses, convex lenses, concave lenses, and combinations thereof. The focus is adjustable by rotating the lens housing of the focusing element 6 by manipulating an adjusting thread. For example, M 12×0.5 type of the focusing lens housing is used, as its thread pitch fully satisfies the application of the apparatus, and such a lens has low acquisition costs. The size and pitch of M 12×0.5 package allows for the adjustment of a focus distance. Several lenses may be used, depending on the image quality for the yarn 1 desired.

The image sensor 3 is formed, for example, by at least one line of light sensitive pixels. Each of the pixels is, for example, a square or rectangular shaped light sensitive area. Individual pixels can be monochromatic or sensitive for specific ranges of wavelengths. The image sensor 3 is, for example, a CCD type or CMOS type, and is formed as one or more lines of photosensitive pixels. Each of the image sensor 3 pixels includes, for example, a micro lens for increased sensitivity and for limiting wide angle light rays, resulting in sharper images.

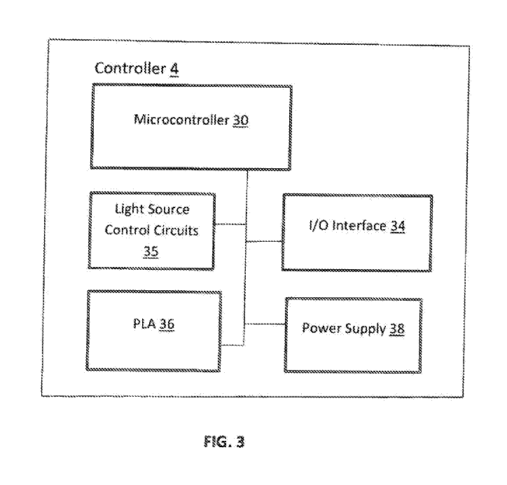

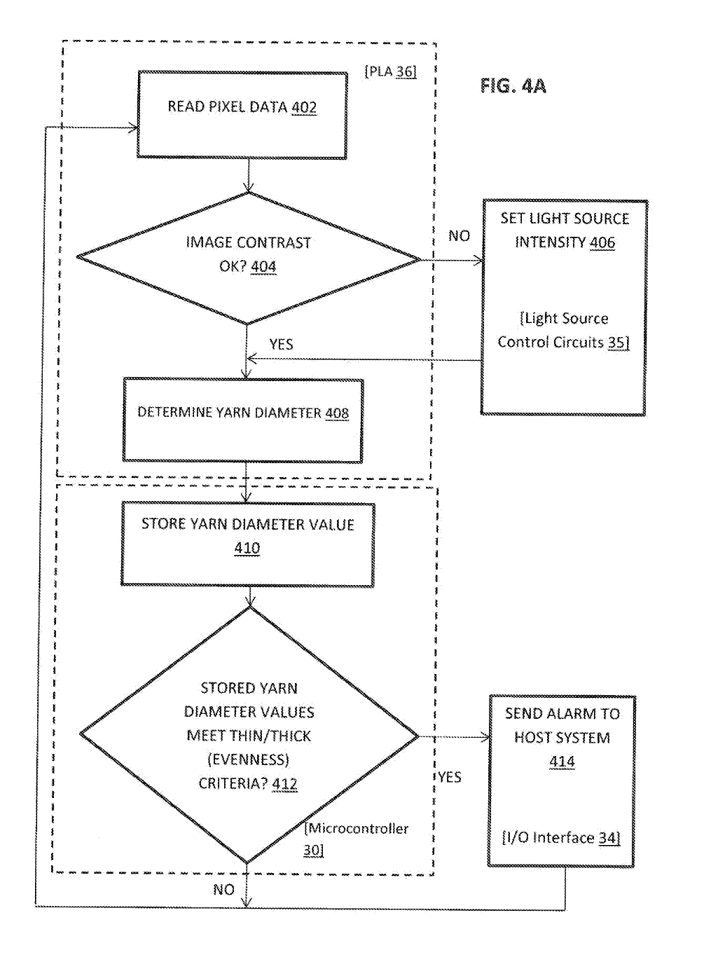

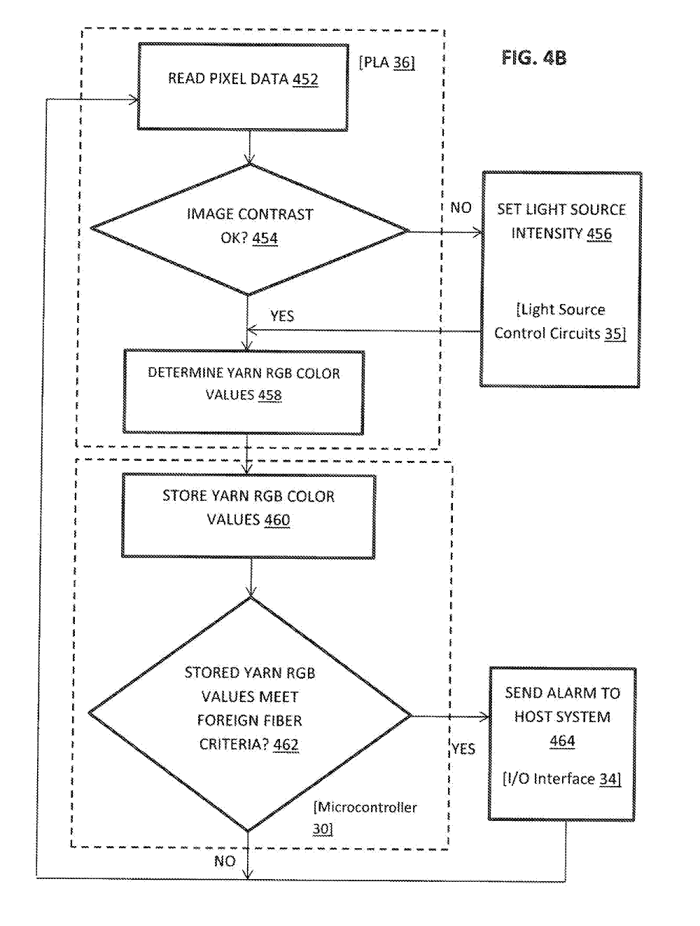

FIG. 3 shows the control unit or controller 4. The controller 4 includes a computer, such as a microcontroller 30, for example, an integrated circuit having a processor core with other embeded functions, such as I/O (input/output) peripherals, data memory, program memory, and the like. The controller 4 also includes an I/O (input/output) interface 34, light source control circuits 35 and a programmable logic array (PLA) module 36. The PLA 36 module functions in yarn diameter calculations and color analysis from captured image data. The program memory of the microcontroller 30 (e.g., ARM, Atmel AVR, Intel 8051, as well as any combinations thereof) stores executable instructions running the algorithms for determining yarn eveness (thick and thin places) (FIG. 4A) and foreign fiber detection (FIG. 4B). The I/O interface module 34 serves for data exchange with a host system. The host system is, for example, a central unit of a spinning machine yarn quality control system (or the central unit of a winding machine yarn quality control system). The host system can be integrated with the central unit of the entire spinning/winding machine, or it can be separate. The host system links to the controller 4 via a communication bus.

The controller 4 also includes light source control circuits 35, which are controlled by the PLA 36. The control unit 4 further includes components, such as power supply 38, and other known in the art for controller operation. For example, the image sensor 3 and functionalities of the control unit 4 can be integrated in an Application Specific Integrated Circuit (ASIC circuit), to simplify the device and provide a low cost solution to yarn quality measurement. All of the components of the controller 4 communicate with each other, directly or indirectly.

The controller 4 is such that the parameters of the primary artificial light source 2 and/or the secondary artificial light source 7 are controlled via feedback to the PLA 36. The feedback is based on the quality of previously captured images from the image sensor 3, as linked to the PLA 36. For example, light intensity is increased (as signalled to the primary 2 and secondary 7 light sources from the light source control circuits module 35), when images with low light intensity were previously captured by the image sensor 3.

Attention is now directed to FIGS. 4A and 4B, which show flow diagrams detailing computer-implemented processes in accordance with embodiments of the disclosed subject matter. Reference is also made to elements shown in FIGS. 1-3. The process and subprocesses of FIGS. 4A and 4B are computerized processes performed by the controller 4. The aforementioned processes and sub-processes can be, for example, performed manually, automatically, or a combination thereof, and, for example, in real time.

In FIG. 4A yarn diameter analysis and determination begins as pixel data (from the image sensor 3), based on light emitted from the primary light source 2, is read by the PLA 36, at block 402, for each yarn segment of a predetermined length. The PLA 36 then applies rules and policies to the read pixel data, to determine whether the image contrast is within a predetermined range, at block 404.

If no at block 404, the process moves to block 406, where the light source intensity, for example, of the primary light source 2, is set to obtain acceptable image contrast in the next measuring cycle, by the light source control circuits 35, at block 406. From block 406, the process moves to block 408. Also, at block 404, should the image contrast be within a predetermined range, the process also moves to block 408. At block 408, the PLA 36 determines the yarn diameter, by processing the data from the pixels by, for example, threshold method, such as binarizing the pixel data and counting pixels corresponding to the yarn 1.

From block 408, the process moves to block 410, where each determined yarn diameter value for captured yarn segment image is stored in the microcontroller 30. It is then determined by the microcontroller 30, at block 412, whether the history of the stored yarn diameter values (currently and previously stored values) fail to meet a predetermined criteria for thickness (e.g., yarn evenness). If yes, the yarn diameter is outside of an acceptable range for several consecutive measured yarn segments (for any number of predetermined segments designating a sample size of the like), e.g., too thin or too thick, and the I/O Interface 34 sends a yarn evenness alarm to the host system, at block 414. From block 414, or if no, at block 412 (the yarn diameter is within a predetermined thickness range, the process returns to block 402, from where it continues.

In FIG. 4B yarn foreign fiber analysis and determination begins as pixel data, based on secondary light source 7 light reflected from the yarn, is read by the PLA 36, at block 452, for each yarn segment of a predetermined length. The PLA 36 then applies rules and policies to the read pixel data, to determine whether the image contrast is within a predetermined range, at block 454.

If no at block 454, the process moves to block 456, where the light source intensity, for example, of the secondary light source 7, is set to result in acceptable image contrast in the next measuring cycle, by the light source control circuits 35, at block 456. From block 456, the process moves to block 458. Also, at block 454, should the image contrast be within a predetermined range, the process also moves to block 458. At block 458, the PLA 36 determines RGB (Red-Green-Blue) color values (so called color analysis) for the yarn segment, for example, by processing light intensities of image sensor 3 pixels at specific positions relative to the position of the yarn image.

From block 458, the process moves to block 460, where each determined RGB color value is stored in data memory associated with the microcontroller 30. It is then determined by the microcontroller 30, whether the history of the stored yarn RGB color values (currently and previously stored values) meets a predetermined criteria for foreign fibers, at block 462. If yes, there are sufficient foreign fibers in the yarn 1 for several consecutive yarn segments to cause the I/O Interface 34 to send an foreign fiber alarm to the host system, at block 464. From block 464, or if no, at block 462, the process returns to block 452, from where it continues.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

The above-described processes including portions thereof can be performed by software, hardware and combinations thereof. These processes and portions thereof can be performed by computers, computer-type devices, workstations, processors, micro-processors, other electronic searching tools and memory and other non-transitory storage-type devices associated therewith. The processes and portions thereof can also be embodied in programmable non-transitory storage media, for example, compact discs (CDs) or other discs including magnetic, optical, etc., readable by a machine or the like, or other computer usable storage media, including magnetic, optical, or semiconductor storage, or other source of electronic signals.

The processes (methods) and systems, including components thereof, herein have been described with exemplary reference to specific hardware and software. The processes (methods) have been described as exemplary, whereby specific steps and their order can be omitted and/or changed by persons of ordinary skill in the art to reduce these embodiments to practice without undue experimentation. The processes (methods) and systems have been described in a manner sufficient to enable persons of ordinary skill in the art to readily adapt other hardware and software as may be needed to reduce any of the embodiments to practice without undue experimentation and using conventional techniques.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. An yarn quality monitoring device, comprising:
a primary light source for emitting light, the light propagation defining an optical path;
a diffuser proximate to the first light source for diffusing the emitted light;
an image sensor illuminated by the diffused light emitted from the primary light source, for capturing a yarn contour image from illumination of a moving yarn in the optical path;
a focusing element in the optical path between the first light source and the image sensor, for focusing the yarn contour image on the image sensor; and
a controller in communication with the image sensor for processing the captured yarn contour image, to determine yarn diameter.

2. The device of claim 1, additionally comprising:
a secondary light source for illuminating the yarn, the reflected light propagation defining an optical path;
a diffraction element in the optical path, between the yarn and the image sensor, the diffraction of secondary light source light reflected from the yarn for interference pattern creation on the image sensor; and
the controller for processing the interference pattern to obtain yarn color analysis.

3. The device of claim 2, wherein the diffraction element is a diffraction grating.

4. The device of claim 2, wherein the primary light source and the secondary light source emit light at different wavelengths, and the device further includes an optical filter at least partially covering the image sensor for blocking light reflected from the yarn illuminated by secondary light source.

5. The device of claim 2, wherein the secondary light source emits one or more light types including monochromatic light, RGB (Red-Green-Blue) light, white light, UV (ultraviolet light) or IR (infra red) light.

6. The device of claim 1, wherein the focusing element includes one or more lenses selected from the group consisting of aspherical lenses, mirror lenses, convex lenses, concave lenses and combinations thereof.

7. The device of claim 1, wherein the focusing element includes one or more lenses in an M12×0.5 package.

8. The device of claim 1, wherein the image sensor comprises:
one or more lines of photosensitive pixels; and
the image sensor is type of a CMOS (complementary metal oxide semiconductor) sensor or a CCD (charge coupled device).

9. The device of claim 1, wherein the image sensor includes microlenses configured to limit wide angle light rays.

10. The device of claim 1, wherein the controller comprises:
a programmable logic array (PLA) for image data processing, to obtain yarn diameter and/or yarn color analysis;
a light control circuit in communication with the PLA, which is controlled by the PLA; and
a microcontroller in communication with the PLA, the microcontroller programmed to evaluate the yarn eveness, and/or the yarn foreign fiber content, from the processed image data.

11. A method of yarn quality monitoring comprising:
illuminating an image sensor with light from a primary light source configured for creating a contour image of a yarn on the image sensor, the primary light source diffused by a diffuser and focused by a focusing element, on an image sensor, the primary light source, diffuser and focusing element aligned to define an optical path;
obtaining a contour image of the yarn by the image sensor, the moving yarn positioned in the optical path between the primary light source and the image sensor; and
processing the yarn contour image to obtain a yarn diameter.

12. The method of claim 11 further comprising:
illuminating the moving yarn by a secondary light source, such that the light from the secondary light source is reflected from the moving yarn;
focusing the reflected light on the image sensor;
diffracting the reflected light from the moving yarn to cause the creation of an interference pattern on the image sensor;
obtaining the interference pattern from the image sensor; and
processing of the interference pattern for obtaining a yarn color analysis.

13. The method of claim 12, wherein the secondary light source emits at least one type of light including: monochromatic light, RGB light, white light, UV light, IR light, and combinations thereof.

14. The method of claim 12, wherein the illuminating by the primary light source is at a wavelength different from a wavelength of light emitted from the secondary light source, and further comprising:
covering a portion of the image sensor with an optical filter;
passing the emitted light from the primary light source through the optical filter to a portion of the image sensor; and
blocking light reflected from the yarn, to cause illumination from only the primary light source for measuring a diameter of the moving yarn.

15. The method of claim 14, wherein the capturing of the contour image of the moving yarn and the capturing of the interference pattern is performed simultaneously by the image sensor.

16. The method of claim 11 additionally comprising:
controlling the primary light source to emit light at an intensity based on processing results from a previously captured image from the image sensor in a feedback loop.

17. The method of claim 12 additionally comprising:
controlling the secondary light source to emit light at an intensity based on processing results from previously captured image from the image sensor in a feedback loop.
